(19) United States
(12) Patent Application Publication
(10) Pub. No.: US 2021/0074397 A1
(43) Pub. Date: Mar. 11, 2021

(54) APPARATUS AND METHOD FOR PROCESSING OF A PRESCRIPTION

(71) Applicant: TAIPEI MEDICAL UNIVERSITY, TAIPEI CITY (TW)

(72) Inventor: YU-CHUAN LI, TAIPEI CITY (TW)

(21) Appl. No.: 16/563,625

(22) Filed: Sep. 6, 2019

(51) Int. Cl. G16H 15/00; G16H 20/10

(52) U.S. Cl. CPC G16H 15/00; G16H 20/10

(57) ABSTRACT

An apparatus and a method for processing a prescription are introduced. The apparatus includes at least one non-transitory computer-readable medium and at least one processor. The at least one non-transitory computer-readable medium and computer executable instructions are configured to, with the at least one processor, cause the apparatus to perform: receiving a first code set comprising at least one diagnosis code and at least one medicine code classification codes; outputting one or more medicine codes of the first code set; receiving a first signal responsive to the output medicine codes; and updating first weights and second weights associated with the output medicine codes in a database in communication with the at least one processor. Therefore, the apparatus and method enhance the accuracy of prescriptions, reduce likelihood of medication errors, cut medical expenditure, and save patients' lives.

### BACKGROUND

### 1. Field of the Disclosure

The present disclosure relates to an apparatus and a method for processing a prescription. More particularly, the present disclosure relates to an apparatus and a method for detecting medication errors.

### 2. Description of the Related Art

Medication errors, which are not uncommon, compromise patients' health or even causing the patients to end up with life-threatening conditions, but also adding to medical expenditure greatly and insidiously. This will happen, if a physician gives no considerations to a patient's condition when prescribing a prescription or in case of cross reaction between medications prescribed by different specialists.

Statistics reported that nearly 100,000 individuals die per year in the United States due to preventable medical errors, most of which are medication errors. In addition, studies also reported 39% of medication errors occur during prescribing; 12% occur during transcribing at the pharmacy; 11% occur during compounding at the pharmacy, and 39% occur during administration as well as only 2% of medication errors are intercepted at some point in the medication administration process. In view of the above, in order to detecting medication errors, there is a need to provide an apparatus and a method for processing a prescription to help physicians and pharmacists to determine the appropriateness of the prescription.

### SUMMARY

To improve the efficiency of detecting medication errors, we used a set of data mining techniques for identifying diagnosis-medication associations and medication-medication associations in order to develop a probabilistic model to processing prescriptions. However, some medications, which may be based on new indications, off-label uses, or physicians' experiences, may be detected as medication errors, and frequent alerts or notifications will occur. Furthermore, some erroneous diagnosis-medication associations (pseudo associations) may occur during data mining. For example, an antihypertensive agent may have high associations with respect to both of hypertension and diabetes because a diabetic often suffer from hypertension.

In one aspect according to some embodiments, an apparatus comprises at least one non-transitory computer-readable medium and at least one processor. The at least one non-transitory computer-readable medium has computer executable instructions stored therein. The at least one processor is coupled to the at least one non-transitory computer-readable medium. The at least one non-transitory computer-readable medium and the computer executable instructions are configured to, with the at least one processor, cause the apparatus to perform: receiving a first code set; outputting one or more medicine codes of the first code set; receiving a first signal responsive to the output medicine codes; and updating first weights and second weights associated with the output medicine codes in a database in communication with the at least one processor. The first code set comprises at least one diagnosis code and at least one medicine code classification codes.

In one aspect according to some embodiments, an apparatus comprises at least one non-transitory computer-readable medium and at least one processor. The at least one non-transitory computer-readable medium has computer executable instructions stored therein. The at least one processor is coupled to the at least one non-transitory computer-readable medium. The at least one non-transitory computer-readable medium and the computer executable instructions are configured to, with the at least one processor, cause the apparatus to perform: receiving a first code set; outputting the diagnosis codes and the medicine codes of the first code set to a database in communication with the at least one processor, wherein; receiving first weights and second weights associated with the diagnosis codes and medicine codes of the first code set from the database; and for each medicine code of the firs code set, saving the first weight with the greatest values with at least one processor. The first code set comprises at least one diagnosis code and at least one medicine code classification codes. The database includes a first weight between a diagnosis code and a medicine code (Q_{DM}) and a second weight between a medicine code and another medicine code (Q_{MM}), and the first weights and the second weights are greater than zero.

In one aspect according to some embodiments, an apparatus comprises at least one non-transitory computer-readable medium and at least one processor. The at least one non-transitory computer-readable medium has computer executable instructions stored therein. The at least one processor is coupled to the at least one non-transitory computer-readable medium. The at least one non-transitory computer-readable medium and the computer executable instructions are configured to, with the at least one processor, cause the apparatus to perform: receiving first weights between diagnosis codes and medicine codes from a database in communication with the at least one processor; and for each medicine code, outputting the first weight with the greatest values to a database in communication with the at least one processor.

In one aspect according to some embodiments, a method for processing a prescription comprises: receiving a first code set; outputting one or more medicine codes of the first code set; receiving a first signal responsive to the output medicine codes; and updating first weights and second weights associated with the output medicine codes in a database. The first code set comprises at least one diagnosis code and at least one medicine code classification codes.

In one aspect according to some embodiments, a method for processing a prescription comprises: receiving a first code set; outputting the diagnosis codes and the medicine codes of the first code set to a database; receiving first weights and second weights associated with the diagnosis codes and medicine codes of the first code set from the database; and for each medicine code of the first code set, saving the first weight with the greatest values. The first code set comprises at least one diagnosis code and at least one medicine code classification codes. The database includes a first weight between a diagnosis code and a medicine code and a second weight between a medicine code and another medicine code, and the first weights and the second weights are greater than zero.

In one aspect according to some embodiments, a method for determining associations between diagnosis codes and medicine codes, comprise: receiving first weights between the diagnosis codes and the medicine codes from a database in communication with the at least one processor; and for each medicine code, outputting the first weight with the greatest values to a database in communication with the at least one processor.

Other aspects and embodiments of the present disclosure are also contemplated. The foregoing summary and the following detailed description are not meant to limit the present disclosure to any particular embodiment but are merely meant to describe some embodiments of the present disclosure.

### BRIEF DESCRIPTION OF THE DRAWINGS

For a better understanding of the nature and objects of some embodiments of the present disclosure, reference should be made to the following detailed description taken in conjunction with the accompanying drawings. In the drawings, identical or functionally identical elements are given the same reference numbers unless otherwise specified.

FIG. 1 illustrates a system according to some embodiments of the present disclosure.

FIG. 2 illustrates associations between diagnoses and medications according to some embodiments of the present disclosure.

FIG. 3 is a flow chart flow chart of validating and testing the present disclosure according to an embodiment of the present invention according to some embodiments of the present disclosure.

FIG. 4 is a flow chart of operations according to some embodiments of the present disclosure.

FIG. 5 is a flow chart of operations according to some embodiments of the present disclosure.

FIG. 6 is a flow chart of operations according to some embodiments of the present disclosure.

FIG. 7 is a flow chart of operations according to some embodiments of the present disclosure.

FIG. 8 is a flow chart of operations according to some embodiments of the present disclosure.

### DETAILED DESCRIPTION

The present disclosure embodies automated techniques for identifying the associations among disease-medication as well as medication-medication and computing their association strength. All diagnosis-medication (DM) and medication-medication (MM) associations, and their association weights, which are derived from medication big data (i.e. prescriptions), are stored in knowledge database. The following example illustrates the knowledge database whose medical big data are borrowed from the Taiwan National Health Insurance Research Database.

The Taiwan's National Health Insurance (NHI) claims data from 2002 with a total of 263.6 million prescriptions is collected. All data are about out-patient visits from hospitals and clinics in Taiwan. Each record (i.e., prescription) consists of the date of visit, patient's pseudo-ID, age, gender, primary diagnosis and secondary diagnosis in NHI codes. Each prescription also includes one to three diagnostic codes and 1 to 15 medicine codes. 160.1 million prescriptions are excluded due to the following reasons: (a) missing or invalid disease codes or medicine codes; and (b) the use of traditional Chinese medicine prescriptions. Thus, the remaining 103.5 million prescriptions with 204.5 million diagnosis ICD-9-CM (International Statistical Classification of Diseases and Related Health Problems, 9th Revision, Clinical Modification) codes and 347.7 million medications with the Taiwan NHI codes are used in the analysis. These medicine codes are mapped to the ATC (Anatomical Therapeutic Chemical) classification code system. The dataset consists of 13,070 unique ICD-9-CM codes and 1,548 unique ATC codes. In some embodiments, diagnosis codes of ICD-10 (International Statistical Classification of Diseases and Related Health Problems, 10th Revision), ICD-10-CM (International Statistical Classification of Diseases and Related Health Problems, 10th Revision, Clinical Modification), or ICD-10-TM (International Statistical Classification of Diseases and Related Health Problems, 10th Revision, Thai Modification) are used.

According to an embodiment of the present invention, the combination of disease-medication and medication-medication are associated as a result of their co-occurrence in a prescription for each patient's visit to a physician. The weights (or association strength values) of disease-medication and medication-mediation is defined the ratios between the joint probability of diagnosis-medication and medication-medication with respect to their expected probability under the independent assumption. The weight is denoted as Q. The weights of disease-medication and medication-mediation are denoted by Q_{DM} and Q_{MM}, respectively.

Based on this definition, each DM and MM pair association strength is computed using 2×2 table (see Table 1). In this table, for a given rule (X→Y), ‘a’ presents the number of transactions in the database containing both X and Y, ‘b’ the number containing X but not Y, ‘c’ the number containing Y but not X, and ‘d’ the number containing neither X nor Y. C1 and C2 are the total number of prescriptions prescribed with disease and medication respectively.

TABLE 1

| | Medication Y | Medication Y | |
|---|---|---|---|
| | present | not present | |
| Diagnosis X present | a | b | a + b (=C1) |
| Diagnosis X not present | c | d | |
| | a + c (=C2) | | N (=a + b + |
| | | | c + d) |

$${Q}_{XY} = \frac{P ( X , Y )}{P ( X ) \times P ( Y )} = \frac{P ( X \vert Y )}{P ( X )} = \frac{\frac{a}{N}}{\frac{( a + b )}{N} \times \frac{( a + c )}{N}} \qquad Formula 1$$

According to Formula 1, Q_{XY} is in the range of [0, +∞]; with Q_{XY}=1 indicates no association between diagnosis and medication, Q_{XY}<1 indicates that diagnosis and medication are negatively associated (i.e. negative Q_{DM}), and Q_{XY}>1 indicates that diagnosis and medication are positively associated (i.e. positive Q_{DM}—the prescriptions with disease X containing medication Y occur more often than other medications). In some embodiments, Q_{XY} is mapped to the range of [0.2, 2].

After processing all observed DM and MM associations, we created a database (term as NHI claims database) that has a total of 1.34 million DM and 0.65 million MM pairs with their Q values (i.e., Q_{DM} and Q_{MM}). The DM and MM associations with less than 5 co-occurrences, by default are considered as “uncommon or rare associations” and are not included in developing the databases. Moreover, according to some embodiments of the present disclosure, the cut-off value for Q is 1. For any association (DM or MM) having Q value less than 1 is regarded as a negative association or uncommon association. The cut-off value can able be adjusted to improve the system.

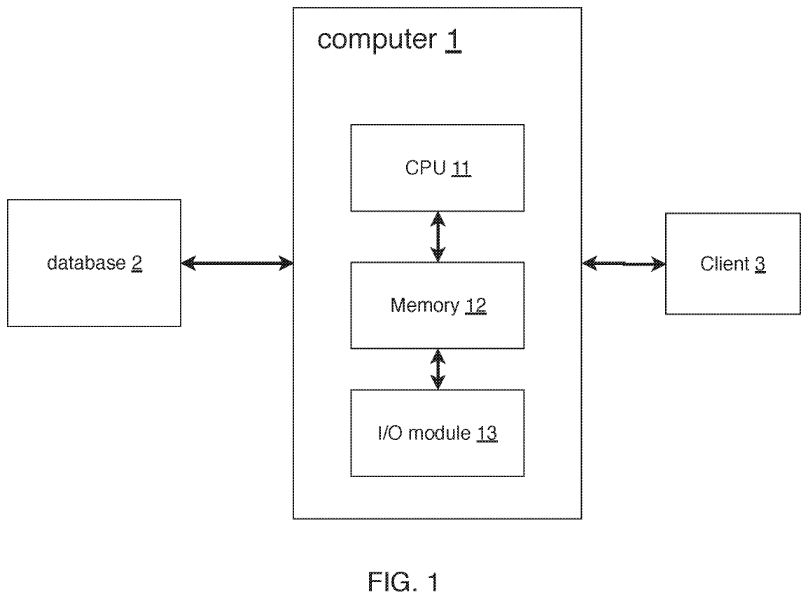

The present disclosure provides a system for processing a prescription. Referring to FIG. 1, the system essentially comprises a computer 1, a database 2, and a client 3. The computer 1, database 2, and the client 3 are in communication. In one embodiment, the computer 1 and the database 2 are in communication, and the computer 1 and the client 3 are in communication.

In some embodiments, the client 3 includes at least one non-transitory computer-readable medium having computer executable instructions stored therein, at least one processor coupled to the at least one non-transitory computer-readable medium, and an input/output module. The at least one non-transitory computer-readable medium and the computer executable instructions are configured to, with the at least one processor, cause the apparatus to perform various operations. In one embodiment, a physician enters a prescription with the client 3. The client 3 may be a personal computer, a smart device, or a portable electronic device. The prescription entered by the physician is transformed into or recorded as a code set. The code set comprises at least one diagnosis code and at least one medicine code. The diagnosis codes may be mapped to one of ICD-9-CM, ICD-10, ICD-10-CM, and ICD-10-TM code systems. The medicine code may be mapped to ATC code system, LOINC (Logical Observation Identifiers Names and Codes) code system, or SNOMED (Systematized Nomenclature of Medicine) code system. The code set is output to the computer 1.

The computer 1 receives the code set. The computer 1 may be a server. In some embodiments, the computer 1 comprises a central processing unit (CPU) 11 (or a processor), a memory 12 (or a non-transitory computer-readable medium), and an input/output module 13. The CPU 11, memory 12, and the input/output module 13 are in communication. In some embodiments, the memory 12 has computer executable instructions stored therein. The memory 12 and the computer executable instructions are configured to, with the CPU 11, cause the computer 1 to perform various operations. The computer 1 outputs the diagnosis codes and medications codes of the code set to the database 2.

In some embodiments, the database 2 includes at least one non-transitory computer-readable medium having computer executable instructions stored therein, at least one processor coupled to the at least one non-transitory computer-readable medium, and an input/output module. The at least one non-transitory computer-readable medium and the computer executable instructions are configured to, with the at least one processor, cause the apparatus to perform various operations. The database 2 receives the code set. The database 2 includes a Q_{DM} value between a diagnosis code and a medicine code and a Q_{MM} value between a medicine code and another medicine code. In some embodiments, the Q_{DM} values and the Q_{MM} values are greater than zero. In some embodiments, the Q_{DM} values and the Q_{MM} values are in the range of [0, +∞]. In some embodiments, the Q_{DM} values and the Q_{MM} values are between 0.2 and 2 (i.e., in the range of [0, 2]). The database 2 outputs the Q_{DM} values and the Q_{MM} values associated with the diagnosis codes and the medicine codes of the received code set to the computer 1.

The computer 1 receives the Q_{DM} values and the Q_{MM} values from the database 2. Based on the Q_{DM} values and the Q_{MM} values for the code set, the computer 1 processes the prescription. In particular, the computer 1 determines whether the prescription satisfies the following criteria:

(1) the total number of the Q_{DM} values greater than the cut-off value and the Q_{MM} values greater than the cut-off value is greater than or equal to the number of medicine codes of the code set;

(2) each diagnosis code of the code set has at least one Q_{DM} value greater than the cut-off value; and

(3) each medicine code of the code set has at least one Q_{DM} value greater than the cut-off value or has at least one Q_{MM} value greater than the cut-off value.

The above three criteria are expressed mathematically as:

$$\begin{matrix}\{ \begin{matrix}\# \{ {Q}_{DiMj} > 1 \} + \# \{ {Q}_{MjMk} > 1 \} \geq m \\ ∀ i : ( ∃ {Q}_{DiMj} : {Q}_{DiMj} \geq α ) \\ ∀ j : [ ( ∃ {Q}_{DiMj} : \# {Q}_{DiMj} \geq α ) ( ∃ {Q}_{MjMk} : \#\# \#\# \#\# {Q}_{MjMk} \geq α ) ]\end{matrix} ( \begin{matrix}i ∈ \{ 1 , 2 … n \} ; \\ j , k ∈ \{ 1 , 2 … \} m\end{matrix} ) & Formula 2\end{matrix}$$

In Formula 2, n is the number of diagnosis codes; m is the number of medications; a is cut-off value. Q_{DiMj} denotes the Q_{DM} value between the i-th diagnosis code and the j-th medicine code; Q_{MjMk} is a Q_{MM} denotes the Q_{MM} value between the j-th medicine code and the k-th medicine code. In the present invention, the cut-off value, α, is set to 1 by default.

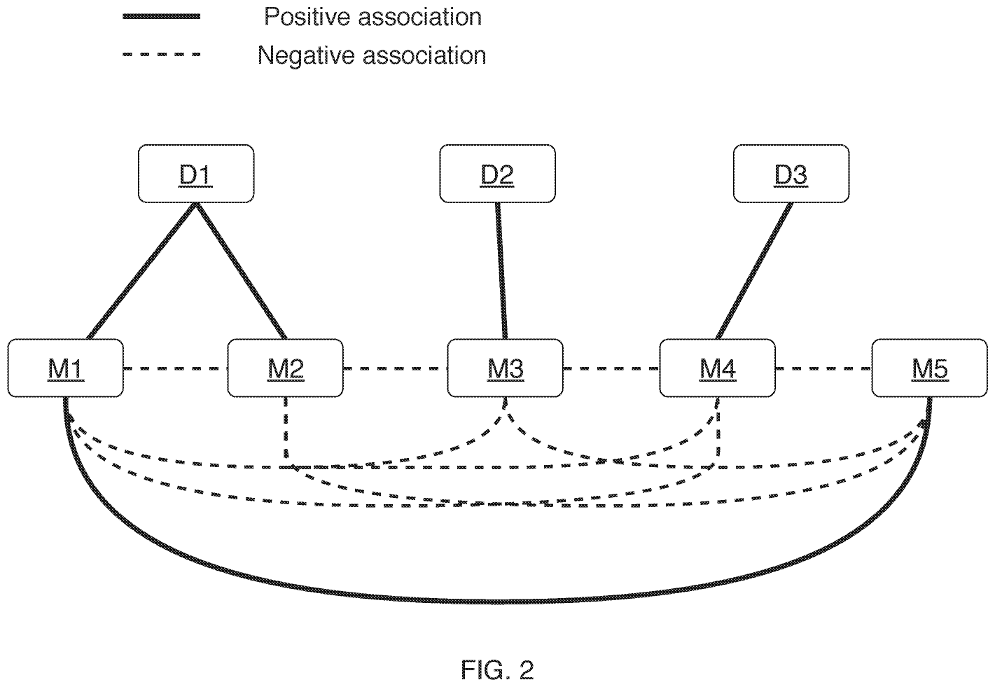

FIG. 2 is a schematic view of an example of a prescription, where D1, D2, and D3 are the diagnosis codes in the prescription, and M1, M2, M3, M4, M5 are the medicine codes. In one embodiment, the computer 1 outputs the diagnosis codes D1, D2, D3, and medicine codes M1, M2, M3, M4, M5 to the databases. The database 2 receives the diagnosis codes D1 to D3 and medications codes M1 to M5 and output the associated Q_{DM} values and Q_{MM} values to the computer 1. In the embodiment of FIG. 2, the database outputs Q_{D1M1}, Q_{D1M2}, Q_{D2M3}, Q_{D3M4}, Q_{M1M2}, Q_{M1M3}, Q_{M1M4}, Q_{M1M5}, Q_{M2M3}, Q_{M2M4}, Q_{M2M5}, Q_{M3M4}, Q_{M3M5}, and Q_{M4M5}. In the embodiment of FIG. 2, Q_{D1M1}, Q_{D1M2}, Q_{D2M3}, Q_{D3M4}, and Q_{M1M5} are positive association, and Q_{M1M2}, Q_{M1M3}, Q_{M1M4}, Q_{M2M3}, Q_{M2M4}, Q_{M2M5}, Q_{M3M4}, Q_{M3M5}, and Q_{M4M5} are negative association. Q_{D1M1}, Q_{D1M2}, Q_{D2M3}, Q_{D3M4}, and Q_{M1M5} are greater the cut-off value (set to 1 by default), and Q_{M1M2}, Q_{M1M3}, Q_{M1M4}, Q_{M2M3}, Q_{M2M4}, Q_{M2M5}, Q_{M3M4}, Q_{M3M5}, and Q_{M4M5} are smaller than the cut-off value (set to 1 by default).

In some embodiments, if the computer 1 determines that the code set satisfies all the three criteria, the computer 1 will not send a signal or an alert to the client 3, for example, to check whether the corresponding prescription is appropriate. In some embodiments, if the computer 1 determines that the code set does not satisfy all the three criteria, the computer 1 will send a signal or an alert to the client 3, for example, to check whether the corresponding prescription is appropriate, and outputs signals to the client 3.

In some embodiments, if the client 3 receives the signals from the computer 1 indicating that the code set does not satisfy all the three criteria, the client 3 alerts or notifies the physician or the pharmacist to check whether the corresponding prescription is appropriate or should be modified.

In the present disclosure was been evaluated by human experts. Firstly, the present disclosure undergoes an initial testing based on the verifying dataset. Subsequently, the system undergoes a second evaluation by human experts including four physicians and three clinical pharmacists to measure the accuracy of the present disclosure.

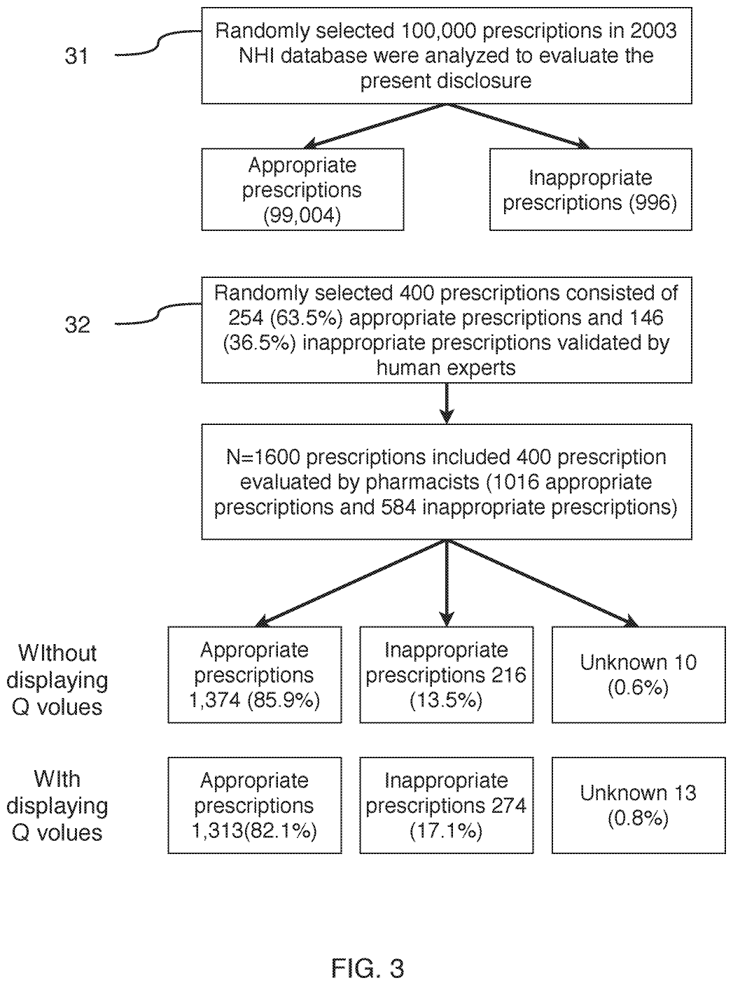

FIG. 3 is a flow chart of validating and testing the present disclosure according to some embodiments of the present disclosure. In operation 31, 100,000 prescriptions are randomly selected from the 2003 NHI database. Afterward, the present disclosure is used to help physicians and pharmacists to test the selected prescriptions for appropriateness. From a total of 100,000 prescriptions, 99,004 prescriptions (99.004%) are identified as appropriate and 996 prescriptions (0.996%) are identified as inappropriate through the present disclosure.

In operation 32, 400 prescriptions are randomly selected out of 100,000 prescriptions which are tested in operation 31. The 400 prescriptions selected to be evaluated by experts (e.g., physicians or pharmacists) include 254 (63.5%) appropriate prescriptions and 146 (36.5%) inappropriate prescriptions. In order to facilitate the identification and measurement of prescriptions, all experts are explained the purpose of the study and are asked to mark whether they agree, disagree or are unsure regarding the overall prescriptions data provided to them. Next, the same prescriptions are re-evaluated with two types of questionnaires—with and without showing Q value for each DM association present in the prescription. The appropriate and inappropriate prescriptions are identified and mixed within the same questionnaire. We administer the questionnaires to four physicians at their clinics (two hundred prescriptions each physician) and three clinical pharmacists (eight hundred prescriptions each pharmacist) at the hospital pharmacies. The questionnaires without Q values are filled out first by all experts, followed by the questionnaires with Q values. Overall, we administer 3,200 prescriptions (1,600 prescriptions without Q values and 1,600 prescriptions with Q values).

The sensitivity, specificity, positive predictive value (PPV), and negative predictive value (NPV) are computed from the results obtained in order to compare the differences and the consensus between the system and experts.

When the Q values are not disclosed, the experts respond to 1,590 (99.3%) prescriptions of which 1,374 (85.9%) are appropriate and 216 (13.5%) are inappropriate prescriptions, leaving 10 prescriptions as “unknown.” However, when the Q value are shown in the prescriptions, the experts respond only to 1,587 (99.2%) prescriptions in which 1,313 (82.1%) are appropriate, 274 (17.1%) are inappropriate prescriptions, and 13 prescriptions are classified as “unknown” (see FIG. 3).

The accuracy of the system assessed by clinical pharmacists and results obtained are shown in Table 2. When the Q are disclosed, the average sensitivity, PPV and NPV are 68.8%, 95.6%, and 24.6% of the pharmacists, respectively. The change found in the questionnaires with showing Q values is different to the change in the questionnaires without showing Q values. However, the average sensitivity, PPV and NPV are 74.3%, 98.7%, and 40.6%, respectively.

TABLE 2

| | Without displaying Q_{DM}, % | | | | With displaying Q_{DM}, % | | | |
|---|---|---|---|---|---|---|---|---|
| experts | Sens | Spec | PPV | NPV | Sens | Spec | PPV | NPV |
| Physicians | 74.3 | 82.7 | 94.8 | 43.1 | 76.7 | 84.9 | 94.8 | 50.3 |
| Pharmacists | 68.8 | 75.0 | 95.6 | 24.6 | 74.3 | 94.2 | 98.7 | 40.6 |
| Overall | 71.5 | 78.9 | 95.2 | 33.9 | 75.5 | 89.5 | 96.7 | 45.5 |

Abbreviations: Sens, sensitivity; Spec, specificity; PPV, positive predictive value; NPV, negative predicive value.
Note:
Confidence intervals (CIs) are small for each parameter and are thus omitted from the reported results.

Based on the results described above, the present disclosure provides a prescription analysis methodology which uses a probabilistic model as an efficient tool in automatically identifying uncommon or rare associations between disease-medication and medication-medication in prescriptions. The present disclosure helps to not only reduce medication errors by alerting physicians to check the identified prescriptions identified, but also improve the patients' safety and the overall quality of health care.

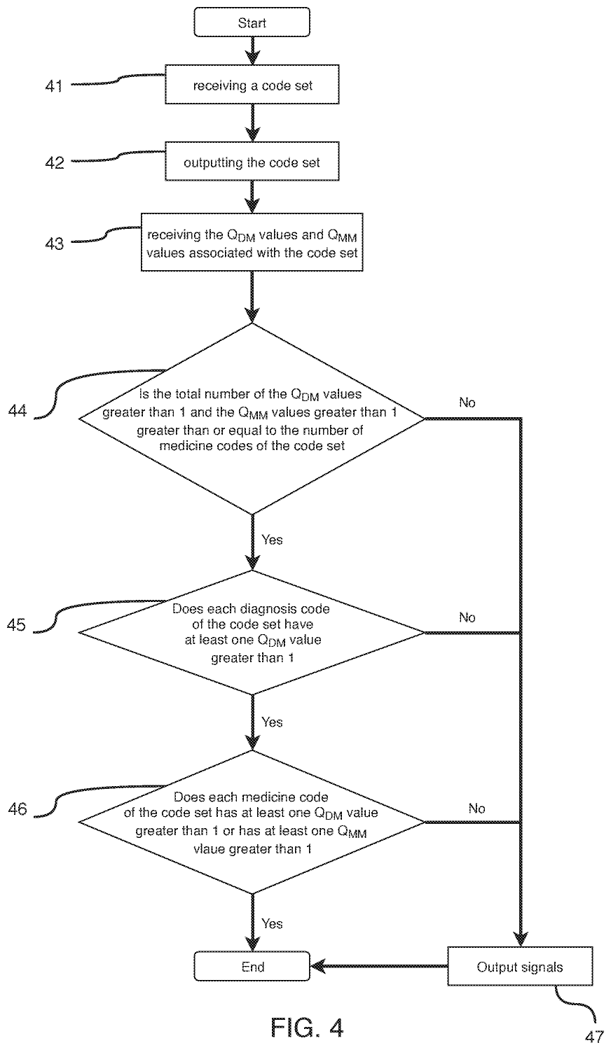

The present disclosure further provides operations performed by the computer 1 for processing a prescription. FIG. 4 is a flow chart of the operations performed by the computer 1 according to some embodiments of the present disclosure. In some embodiments, the memory 12 has computer executable instructions stored therein. The memory 12 and the computer executable instructions are configured to, with the CPU 11, cause the computer 1 to perform the operations.

In operation 41, the computer 1 receives a code set. The code set comprises at least one diagnosis code and at least one medicine code. In some embodiments, the code set is received from the client 3 on which a physician enters prescription. The diagnosis codes may be mapped to one of ICD-9-CM, ICD-10, ICD-10-CM, and ICD-10-TM code systems. The medicine code may be mapped to ATC code system, LOINC (Logical Observation Identifiers Names and Codes) code system, or SNOMED (Systematized Nomenclature of Medicine) code system.

In operation 42, the computer 1 outputs the received code set to the database 2. The database 2 includes a Q_{DM} value between a diagnosis code and a medicine code and a Q_{MM} value between a medicine code and another medicine code. In some embodiments, the database 2 look up the associated Q_{DM} values and Q_{MM} values according to the input diagnosis codes and medications codes and outputs the same.

In operation 43, the computer 1 receives the Q_{DM} values and the Q_{MM} values associated with the code set which was output to the database 2.

In operations 44, 45, and 46, the computer 1 determines whether the received code set satisfy criteria. In particular, the computer 1 determines whether the Q_{DM} values and the Q_{MM} values associated with the code set satisfy all the following three criteria:

(1) the total number of the Q_{DM} values greater than the cut-off value and the Q_{MM} values greater than the cut-off value greater than or equal to the number of medicine codes of the code set;

(2) each diagnosis code of the code set has at least one Q_{DM} value greater than the cut-off value; and

(3) each medicine code of the code set has at least one Q_{DM} value greater than the cut-off value or has at least one Q_{MM} value greater than the cut-off value (the cut-off value is set 1 by default).

In some embodiments, determining whether the Q_{DM} values and the Q_{MM} values associated with the code set satisfy all the following three criteria (i.e., operations 44, 45, and 46) may be exploited by a flag. The computer 1 generates a flag. The computer 1 sets the flag equal to 0 by default. The computer 1 compares the total number of the Q_{DM} values greater than the cut-off value and the Q_{MM} values greater than the cut-off value to the number of medicine codes of the code set. If the total number of the first weights for the first code set greater than a cut-off value and the second weights for the first code set greater than the cut-off value is smaller than the number of medication codes of the first code set, the computer 1 sets the flag equal to 1. The computer 1, for each diagnosis code of the code set, compares the Q_{DM} values to the cut-off value. If the Q_{DM} values of the diagnosis codes of the code set are smaller than or equal to the cut-off value, the computer 1 sets the flag equal to 1. The computer 1, for each medicine code of the code set, compares the Q_{DM} values to the cut-off value and compares the Q_{MM} values to the cut-off value. If the Q_{DM} values and the Q_{MM} values of the medicine code of the code set are smaller than or equal to the cut-off value, the computer 1 set the flag equal to 1. If the flag equals to 0, the Q_{DM} values and the Q_{MM} values associated with the code set satisfy all the three criteria above. If the flag equals to 1, the Q_{DM} values and the Q_{MM} values associated with the code set do not satisfy all the three criteria above.

If the Q_{DM} values and the Q_{MM} values associated with the code set satisfy all the three criteria above, the computer 1 would not output signals. If the Q_{DM} values and the Q_{MM} values associated with the code set do not satisfy all the three criteria above, the computer 1 outputs signals in operation 47. In some embodiments, the computer 1 outputs signals to the client 3 in operation 47. In some embodiments, if the client 3 receives the signals from the computer 1 indicating that the code set do not satisfy all the three criteria, the client 3 alerts or notifies the physician to check whether the corresponding prescription is appropriate or should be modified.

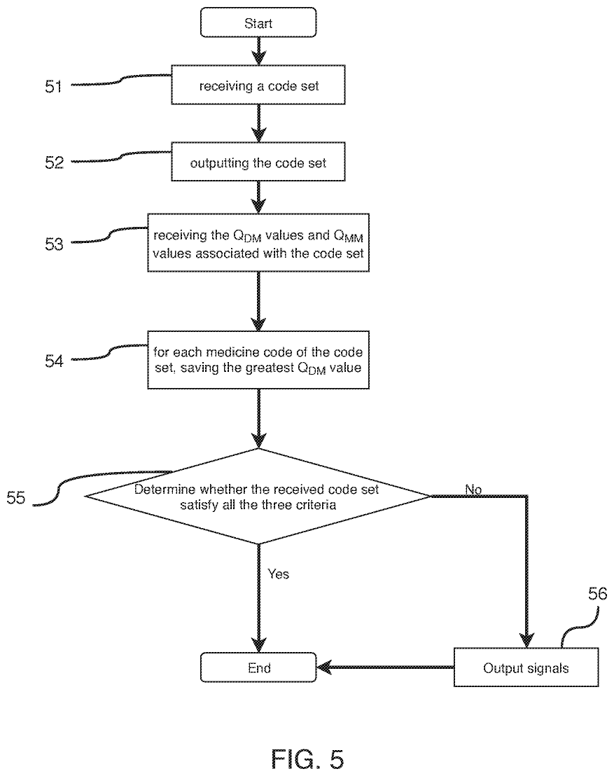

FIG. 5 is a flow chart of the operation performed by the computer 1 according to some embodiments of the present disclosure. In some embodiments, the memory 12 has computer executable instructions stored therein. The memory 12 and the computer executable instructions are configured to, with the CPU 11, cause the computer 1 to perform the operations.

In operation 51, the computer 1 receives a code set. The code set comprises at least one diagnosis code and at least one medicine code. In some embodiments, the code set is received from the client 3 on which a physician enters prescription. The diagnosis codes may be mapped to one of ICD-9-CM, ICD-10, ICD-10-CM, and ICD-10-TM code systems. The medicine codes may be mapped to ATC code system, LOINC (Logical Observation Identifiers Names and Codes) code system, or SNOMED (Systematized Nomenclature of Medicine) code system.

In operation 52, the computer 1 outputs the received code set to the database 2. The database 2 includes a Q_{DM} value between a diagnosis code and a medicine code and a Q_{MM} value between a medicine code and another medicine code. In some embodiments, the database 2 look up the associated Q_{DM} values and Q_{MM} values according to the input diagnosis codes and medications codes and outputs the same.

In operation 53, the computer 1 receives the Q_{DM} values and the Q_{MM} values associated with the code set which was output to the database 2.

During generating Q_{DM} values and Q_{MM} values from or mining in the prescription database (e.g., NHI claims database), some erroneous diagnosis-medication associations (pseudo associations) may occur. For example, an antihypertensive agent may have high associations with respect to both of hypertension and diabetes because a diabetic often suffer from hypertension. Thus, the antihypertensive agent often exists in the prescription with the diagnoses of hypertension and diabetes. Due to this reason, the antihypertensive agent has a high Q_{DM} value with respect to hypertension and has a high Q_{DM} value with respect to diabetes. However, the antihypertensive agent and diabetes should not have a high Q_{DM} value. The high association between the antihypertensive agent and diabetes is called pseudo association.

In operation 54, for each medicine code of the code set, the computer 1 only saves the greatest Q_{DM} value and discard the other Q_{DM} values (or only saving the association with the greatest Q_{DM} value). For the example of a prescription having a antihypertensive agent, hypertension, and diabetes, because not every diabetic suffers from hypertension, although the antihypertensive agent has a high Q_{DM} value with respect to hypertension and has a high Q_{DM} value with respect to diabetes, the Q_{DM} value between the antihypertensive agent and hypertension is greater than that between the antihypertensive agent and diabetes. After operation 54, the pseudo association between the antihypertensive agent and diabetes is removed.

According to some embodiments, in operation 54, for each medicine code of the code set, the computer 1 not only saves the greatest Q_{DM} value but also saves the Q_{DM} value associated to the diagnosis codes in the same group of the diagnosis code for the greatest Q_{DM} value. For the example of a prescription having a antihypertensive agent, essential hypertension, benign essential hypertension, and diabetes, because the Q_{DM} value between the antihypertensive agent and essential hypertension (e.g., diagnosis codes 401 in ICD-9-CM code system) is the greatest, the greatest Q_{DM} value between the antihypertensive agent and essential hypertension is saved, and the Q_{DM} value associated with benign essential hypertension which is in the same group as essential hypertension (e.g., diagnosis code 4011, benign essential hypertension) is save as well.

In some embodiments, in operation 54, for each medicine code, the computer 1 not only saves the association with the greatest Q_{DM} value but also saves the associations having the Q_{DM} values, which are associated with the diagnosis codes in the same group of the diagnosis code for the greatest Q_{DM} value. For the example of a prescription having a antihypertensive agent, essential hypertension, benign essential hypertension, and diabetes, because the Q_{DM} value between the antihypertensive agent and essential hypertension (e.g., diagnosis codes 401 in ICD-9-CM code system) is the greatest, the association of the greatest Q_{DM} value between the antihypertensive agent and essential hypertension is saved, and the association with the Q_{DM} value associated with benign essential hypertension, which is in the same group as essential hypertension (e.g., diagnosis code 4011, benign essential hypertension), is save as well.

In operation 55, the computer 1 determines whether the received code set satisfy all the three criteria as disclosed in operations 44, 45, and 46. In particular, operation 55 comprises operations 44, 45, and 46 shown in FIG. 4. In operation 55, if the computer 1 determines the saved Q_{DM} values and the Q_{MM} values associated with the code set satisfy all the three criteria as disclosed in operations 44, 45, and 46 shown in FIG. 4, the computer 1 would not output signals.

In operation 55, if the computer 1 determines the saved Q_{DM} values and the Q_{MM} values associated with the code set do not satisfy all the three criteria as disclosed in operations 44, 45, and 46 shown in FIG. 4, the computer 1 outputs signals in operation 56. In some embodiments, the computer 1 outputs signals to the client 3 in operation 56. In some embodiments, if the client 3 receives the signals from the computer 1 indicating that the code set do not satisfy all the three criteria, the client 3 alerts or notifies the physician to check whether the corresponding prescription is appropriate or should be modified.

Table 3 shows five exemplary prescriptions for calculating Q_{DM} values according to Table 1 and Formula 1. Table 4 shows the Q_{DM} values between essential hypertension (i.e., diagnosis code (ICD9) 4019) and medications for diabetes (i.e., medicine code (ATC code) A10AB03, A10AB04, A10AC01, A10AD05, A10AE05, A10BA02, A10BA03, A10BB01, A10BB02, and A10BB07). Table 4 shows that the associations between essential hypertension and medications for diabetes are highly positive. The associations between essential hypertension and medications for diabetes shown in Table 4 are called pseudo associations. The processing of the prescriptions would be erroneous if the pseudo associations shown in Table 4 are not removed.

TABLE 3

| | |
|---|---|
| Prescription 1 | Diagnosis: {hypertension} |
| | Medication: {lisinopril, multivitamin} |
| Prescription 2 | Diagnosis: {diabetes, hypertension} |
| | Medication: {insulin, metformin, lisinopril} |
| Prescription 3 | Diagnosis: {diabetes} |
| | Medication: {insulin} |
| Prescription 4 | Diagnosis: {diabetes} |
| | Medication: {metformin} |
| Prescription 5 | Diagnosis: {polycystic ovarian syndrome} |
| | Medication: {metformin} |

TABLE 4

| Diagnosis code (ICD9) | Medicine code (ATC code) | Q_{DM} |
|---|---|---|
| 4019 | A10AB03 | 5.42 |
| 4019 | A10AB04 | 2.67 |
| 4019 | A10AC01 | 4.24 |
| 4019 | A10AD05 | 4.23 |
| 4019 | A10AE05 | 4.84 |
| 4019 | A10BA02 | 5.14 |
| 4019 | A10BA03 | 4.06 |
| 4019 | A10BB01 | 4.82 |
| 4019 | A10BB02 | 5.85 |
| 4019 | A10BB07 | 4.97 |

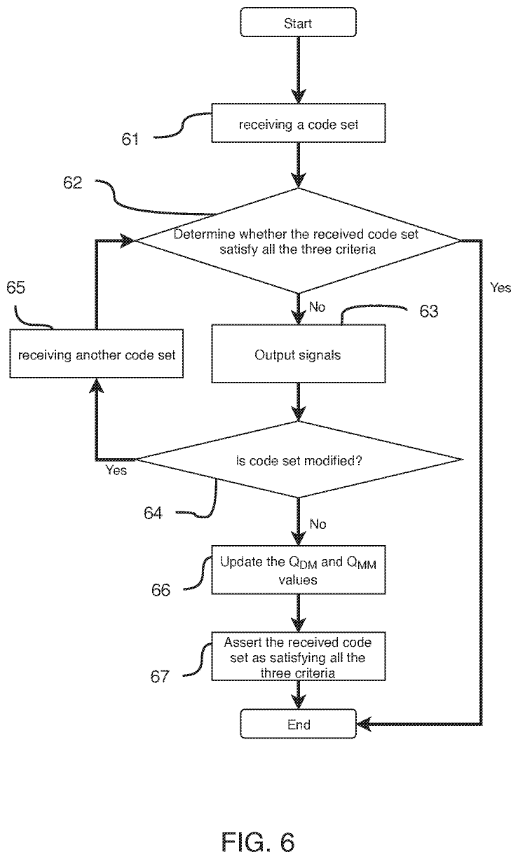

FIG. 6 is a flow chart of the operations performed by the computer 1 according to some embodiments of the present disclosure. In some embodiments, the memory 12 has computer executable instructions stored therein. The memory 12 and the computer executable instructions are configured to, with the CPU 11, cause the computer 1 to perform the operations.

In operation 61, the computer 1 receives a code set. The code set comprises at least one diagnosis code and at least one medicine code. In some embodiments, the code set is received from the client 3 on which a physician enters prescription. The diagnosis codes may be mapped to one of ICD-9-CM, ICD-10, ICD-10-CM, and ICD-10-TM code systems. The medicine codes may be mapped to ATC code system, LOINC (Logical Observation Identifiers Names and Codes) code system, or SNOMED (Systematized Nomenclature of Medicine) code system.

In operation 62, the computer 1 determines whether the received code set satisfy all the three criteria as disclosed in operations 44, 45, and 46. In particular, operation 62 comprises operations 42, 43, 44, 45, and 46 shown in FIG. 4. In operation 62, the computer 1 outputs the received code set to the database 2, receives the Q_{DM} values and the Q_{MM} values associated with the code set which was output to the database 2, and determines whether the saved Q_{DM} values and the Q_{MM} values associated with the code set satisfy all the three criteria of operation 44, 45, and 46 shown in FIG. 4. In operation 62, if the computer 1 determines the saved Q_{DM} values and the Q_{MM} values associated with the code set satisfy all the three criteria as disclosed in operations 44, 45, and 46 shown in FIG. 4, the computer 1 would not output signals.

In operation 62, if the computer 1 determines that the received code set do not satisfy all the three criteria as disclosed in operations 44, 45, and 46 shown in FIG. 4, the computer 1 outputs signals in operation 63. In some embodiments, the computer 1 outputs signals to the client 3 in operation 63. In some embodiments, the computer 1 outputs at least one medicine code to the client 3. The at least one medicine code output from the computer 1 to the client 3 has least one Q_{DM} value smaller than the cut-off value (set to 1 by default) or has least one Q_{MM} value smaller than the cut-off value (set to 1 by default). That is, the at least one medicine code output by the computer 1 has at least one negative association with one diagnosis code of the received code set or has at least one negative association with another medicine code of the received code set. In some embodiments, if the client 3 receives the signals from the computer 1 indicating that the received code set do not satisfy all the three criteria as disclosed in operations 44, 45, and 46 shown in FIG. 4, the client 3 notifies or alerts the physician to check whether the corresponding prescription is appropriate or should be modified. In some embodiments, if the client 3 receives the at least one medicine code from the computer 1 indicating that the received code set do not satisfy all the three criteria as disclosed in operations 44, 45, and 46 shown in FIG. 4, the client 3 notifies (or alerts) the physician the at least one medicine code which may be modified and notifies (or alerts) the physician to check whether the corresponding medicine is appropriate or should be modified.

In operation 64, the computer 1 determines whether the code set (which do not satisfy all the three criteria) is modified. In particular, if the code set (which do not satisfy all the three criteria) is not modified (e.g., on the client 3) within a period of time, the computer 1 receives a signal of first type. Otherwise, if the code set (which do not satisfy all the three criteria) is modified (e.g., on the client 3) within a period of time, the computer 1 receives a signal of second type.

If the computer 1 receives a signal of second type in operation 64, the computer 1 performs operation 65 to receive another code set (e.g., from the client 3). After operation 65, the computer 1 performs operation 62 to determine whether the code set received in operation 65 satisfy all the three criteria as disclosed in operations 44, 45, and 46.

If the computer 1 receives a signal of first type in operation 64, the computer 1 performs operation 66 to updating the Q_{DM} values and Q_{MM} values associated with the output medicine codes output in operation 63. In particular, the computer 1 updates the Q_{DM} values and Q_{MM} values in (or stored in) the database 2 associated with the output medicine codes output in operation 63. In some embodiments, the computer 1 updates the Q_{DM} values and Q_{MM} values smaller than the cut-off value (set to 1 by default) associated with the output medicine codes output in operation 63.

In some embodiments, the Q_{DM} values and Q_{MM} values is updated by multiplying the Q_{DM} values and Q_{MM} values by a number. In some embodiments, the number to be multiplied is larger than 1. In some embodiments, the Q_{DM} values and Q_{MM} values is updated by adding the Q_{DM} values and Q_{MM} values with a number. In some embodiments, the number to be added is larger than 0.

Because some medications are based on new indications, off-label uses, or physicians' experiences, these medications may be determined as not satisfying all the three criteria as disclosed in operations 44, 45, and 46 shown in FIG. 4, and frequent alerts or notifications will occur. The physician responsible for the prescription has checked the alert or notification generated by the present disclosure, and the physician decides not to modify the prescription. The Q_{DM} values and Q_{MM} values associated with the medicine codes which output in operation 63 are updated to avoid frequent alert or notifications.

In operation 67, the computer 1 assert the received codes as satisfying all the three criteria as disclosed in operations 44, 45, and 46 shown in FIG. 4. Because the physician has checked the alert or notification indicative of that the code set (i.e., the prescription) do not satisfy all the three criteria and decides not to modify the prescription, the code set (i.e., the prescription) has been double checked by the physician. Thus, it is trusted to assert the received code set as satisfying all the three criteria in operation 67.

Verifications for the operations shown in FIG. 6 were performed with 938 notifications (e.g., the signal sent in operation 63) and 12 experts (e.g., physicians or pharmacist). In the result, for every three experts, there is one expert agrees the notifications at a percentage of 85%. Please note that, without the operations of updating the Q_{DM} and Q_{MM} values (e.g., operations 66 and 67), an expert agrees the notifications at a percentage lower than 30%.

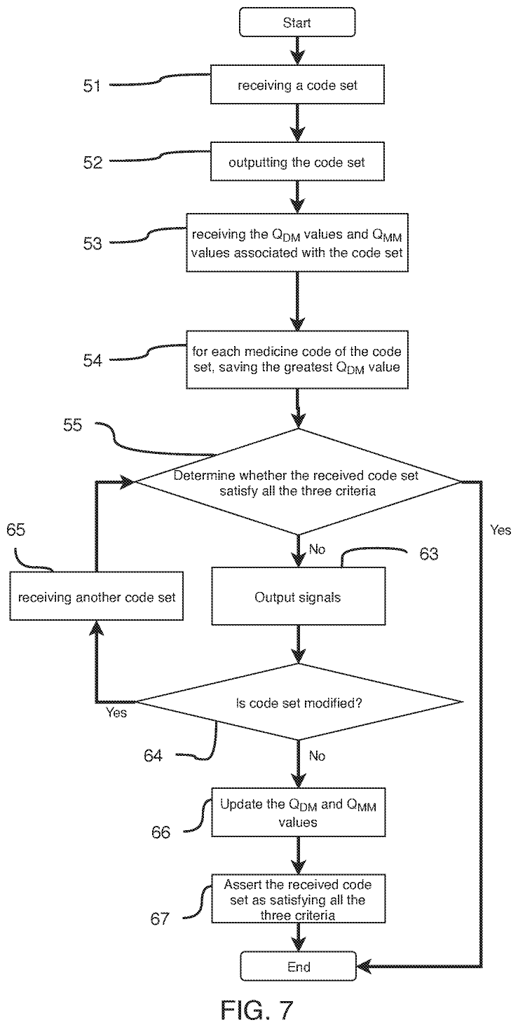

FIG. 7 is a flow chart of the operations performed by the computer 1 according to some embodiments of the present disclosure. In some embodiments, the memory 12 has computer executable instructions stored therein. The memory 12 and the computer executable instructions are configured to, with the CPU 11, cause the computer 1 to perform the operations.

The operations shown in FIG. 7 are a combination of the operations shown in FIGS. 5 and 6. In particular, operations 51, 52, 53, 54, and 55 in FIG. 5 and operations 63, 64, 65, 66, and 67 are applied FIG. 7. For simplicity, the details of FIG. 7 are not described here, please refer to disclosure of operations 51, 52, 53, 54, 55, 63, 64, 65, 66, and 67 above. Based on the disclosure of operations 51, 52, 53, 54, 55, 63, 64, 65, 66, and 67 above, persons skilled in the art should understand the operations shown in FIG. 7.

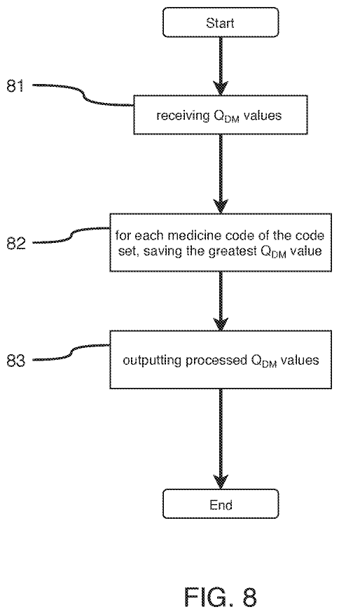

FIG. 8 is a flow chart of the operation performed by the computer 1 according to some embodiments of the present disclosure. In some embodiments, the memory 12 has computer executable instructions stored therein. The memory 12 and the computer executable instructions are configured to, with the CPU 11, cause the computer 1 to perform the operations.

In some embodiments, the operations shown in FIG. 8 are performed after the calculations of the weights of disease-medication (Q_{DM} values) and the weights of medication-mediation (Q_{MM} values) as disclosed in paragraphs 0020-0023. In some embodiments, the operations shown in FIG. 8 can be used to further process the Q_{DM} values and Q_{MM} values stored in the database 2.

During generating Q_{DM} values and Q_{MM} values from or mining in the prescription database (e.g., NHI claims database), some erroneous diagnosis-medication associations (pseudo associations) may occur. For example, an antihypertensive agent may have high associations with respect to both of hypertension and diabetes because a diabetic often suffer from hypertension. Thus, the antihypertensive agent often exists in the prescription with the diagnoses of hypertension and diabetes. Due to this reason, the antihypertensive agent has a high Q_{DM} value with respect to hypertension and has a high Q_{DM} value with respect to diabetes. However, the antihypertensive agent and diabetes should not have a high Q_{DM} value. The high association between the antihypertensive agent and diabetes is called pseudo association.

In operation 81, the computer 1 receives Q_{DM} values. In some embodiments, the diagnosis codes may be mapped to one of ICD-9-CM, ICD-10, ICD-10-CM, and ICD-10-TM code systems, and the medicine codes may be mapped to ATC code system, LOINC (Logical Observation Identifiers Names and Codes) code system, or SNOMED (Systematized Nomenclature of Medicine) code system.

In operation 82, for each medicine code, the computer 1 only saves the greatest Q_{DM} value and discard the other Q_{DM} values (or only saving the association with the greatest Q_{DM} value). For the example of a prescription having a antihypertensive agent, hypertension, and diabetes, because not every diabetic suffers from hypertension, although the antihypertensive agent has a high Q_{DM} value with respect to hypertension and has a high Q_{DM} value with respect to diabetes, the Q_{DM} value between the antihypertensive agent and hypertension is greater than that between the antihypertensive agent and diabetes. After operation 82, the pseudo association between the antihypertensive agent and diabetes is removed.

According to some embodiments, in operation 82, for each medicine code, the computer 1 not only saves the greatest Q_{DM} value but also saves the Q_{DM} values, which are associated with the diagnosis codes in the same group of the diagnosis code for the greatest Q_{DM} value. For the example of a prescription having a antihypertensive agent, essential hypertension, benign essential hypertension, and diabetes, because the Q_{DM} value between the antihypertensive agent and essential hypertension (e.g., diagnosis codes 401 in ICD-9-CM code system) is the greatest, the greatest Q_{DM} value between the antihypertensive agent and essential hypertension is saved, and the Q_{DM} value associated with benign essential hypertension, which is in the same group as essential hypertension (e.g., diagnosis code 4011, benign essential hypertension), is save as well.

In some embodiments, in operation 82, for each medicine code, the computer 1 not only saves the association with the greatest Q_{DM} value but also saves the associations having the Q_{DM} values, which are associated with the diagnosis codes in the same group of the diagnosis code for the greatest Q_{DM} value. For the example of a prescription having a antihypertensive agent, essential hypertension, benign essential hypertension, and diabetes, because the Q_{DM} value between the antihypertensive agent and essential hypertension (e.g., diagnosis codes 401 in ICD-9-CM code system) is the greatest, the association of the greatest Q_{DM} value between the antihypertensive agent and essential hypertension is saved, and the association of the Q_{DM} value associated with benign essential hypertension, which is in the same group as essential hypertension (e.g., diagnosis code 4011, benign essential hypertension), is save as well.

In operation 83, the processed Q_{DM} values are output to the database 2 and stored in the database 2.

After operations 81-83, the pseudo associations in the Q_{DM} values are removed. The processed Q_{DM} values and the Q_{MM} values stored to the database 2 can be used for the operations shown in FIG. 4 or in FIG. 6.

Table 5 shows five exemplary prescriptions for calculating Q_{DM} values according to Table 1 and Formula 1. Without operations shown in FIG. 8, Table 6 shows the Q_{DM} values between essential hypertension (i.e., diagnosis code (ICD9) 4019) and medications for diabetes (i.e., medicine code (ATC code) A10AB03, A10AB04, A10AC01, A10AD05, A10AE05, A10BA02, A10BA03, A10BB01, A10BB02, and A10BB07). Table 6 shows that the associations between essential hypertension and medications for diabetes are highly positive. The associations between essential hypertension and medications for diabetes shown in Table 6 are called pseudo associations.

After the operations shown in FIG. 8, Table 7 shows the Q_{DM} values between essential hypertension (i.e., diagnosis code (ICD9) 4019) and medications for diabetes (i.e., medicine code (ATC code) A10AB01, A10AC01, A10AD01, A10AD05, A10BA02, A10BA03, A10BB01, A10BB02, A10BB05, and A10BB07). Table 7 shows that the associations between essential hypertension and medications for diabetes are negative. The pseudo associations between essential hypertension and medications for diabetes shown in Table 6 are removed.

TABLE 5

| | |
|---|---|
| Prescription 1 | Diagnosis: {hypertension} |
| | Medication: {lisinopril, multivitamin} |
| Prescription 2 | Diagnosis: {diabetes, hypertension} |
| | Medication: {insulin, metformin, lisinopril} |
| Prescription 3 | Diagnosis: {diabetes} |
| | Medication: {insulin} |
| Prescription 4 | Diagnosis: {diabetes} |
| | Medication: {metformin} |
| Prescription 5 | Diagnosis: {polycystic ovarian syndrome} |
| | Medication: {metformin} |

TABLE 6

| Diagnosis code (ICD9) | Medicine code (ATC code) | Q_{DM} |
|---|---|---|
| 4019 | A10AB03 | 5.42 |
| 4019 | A10AB04 | 2.67 |
| 4019 | A10AC01 | 4.24 |
| 4019 | A10AD05 | 4.23 |
| 4019 | A10AE05 | 4.84 |
| 4019 | A10BA02 | 5.14 |
| 4019 | A10BA03 | 4.06 |
| 4019 | A10BB01 | 4.82 |
| 4019 | A10BB02 | 5.85 |
| 4019 | A10BB07 | 4.97 |

TABLE 7

| Diagnosis code (ICD9) | Medicine code (ATC code) | Q_{DM} |
|---|---|---|
| 4019 | A10AB03 | 0.04 |
| 4019 | A10AB04 | 0.06 |
| 4019 | A10AC01 | 0.06 |
| 4019 | A10AD05 | 0.06 |
| 4019 | A10AE05 | 0.23 |
| 4019 | A10BA02 | 0.03 |
| 4019 | A10BA03 | 0.15 |
| 4019 | A10BB01 | 0.11 |
| 4019 | A10BB02 | 0.15 |
| 4019 | A10BB07 | 0.19 |

As used herein, the singular terms “a,” “an,” and “the” may include plural referents unless the context clearly indicates otherwise. For example, reference to an electronic device may include multiple electronic devices unless the context clearly indicates otherwise.

As used herein, the terms “connect,” “connected,” “connection,” “communication”, and “in communication” refer to an operational coupling or linking. Components in communication can be directly or indirectly coupled or linked to one another through, for example, another set of components.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other embodiments of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless otherwise specifically indicated herein, the order and grouping of the operations are not limitations of the present disclosure.

What is claimed is:

1. An apparatus, comprising:
at least one non-transitory computer-readable medium having computer executable instructions stored therein; and
at least one processor coupled to the at least one non-transitory computer-readable medium,
wherein the at least one non-transitory computer-readable medium and the computer executable instructions are configured to, with the at least one processor, cause the apparatus to perform:
receiving a first code set, the first code set comprising at least one diagnosis code and at least one medication code;
outputting one or more medication codes of the first code set;
receiving a first signal responsive to the output medication codes; and
updating first weights and second weights associated with the output medication codes in a database in communication with the at least one processor.

2. The apparatus of claim 1, wherein the first signal is received if the first code set is not modified within a period of time.

3. The apparatus of claim 1, wherein the database includes a first weight between a diagnosis code and a medication code and a second weight between a medication code and another medication code, and the first weights and the second weights are greater than zero.

4. The system of claim 3, wherein:
the output medication codes has at least one first weight smaller than a cut-off value or has at least one second weight smaller than the cut-off value.

5. The apparatus of claim 3, wherein the apparatus is further caused to perform:
generating a flag;
setting the flag to 0;
comparing the total number of the first weights for the first code set greater than a cut-off value and the second weights for the first code set greater than the cut-off value to the number of medication codes of the first code set;
setting a flag equal to 1 if the total number of the first weights for the first code set greater than a cut-off value and the second weights for the first code set greater than the cut-off value is smaller than the number of medication codes of the first code set;
for each diagnosis code of the first code set, comparing the first weights to the cut-off value;
setting the flag equal to 1 if the first weights of the diagnosis codes of the first code set are smaller than or equal to the cut-off value;
for each medication code of the first code set, comparing the first weights to the cut-off value and comparing second weights to the cut-off value; and
setting the flag equal to 1 if the first weights and second weights of the medicine codes of the first code set are smaller than or equal to the cut-off value.

6. The system of claim 5, wherein:
the apparatus is further caused to perform, if the flag equals to 1, outputting one or more medication codes of the first code set.

7. The apparatus of claim 5, wherein the apparatus is further caused to perform:
receiving a second signal responsive to the output medication codes;
receiving a second code set comprising at least one diagnosis codes and at least one medication codes;
setting the flag to 0;
comparing the total number of the first weights for the second code set greater than a cut-off value and the second weights for the second code set greater than the cut-off value to the number of medicine codes of the second code set;
setting a flag equal to 1 if the total number of the first weights for the second code set greater than a cut-off value and the second weights for the second code set greater than the cut-off value is smaller than the number of medicine codes of the second code set;
for each diagnosis code of the second code set, comparing the first weights to the cut-off value;
setting the flag equal to 1 if the first weights of the diagnosis codes of the second code set are smaller than or equal to the cut-off value;
for each medicine code of the second code set, comparing the first weights to the cut-off value and comparing second weights to the cut-off value; and
setting the flag equal to 1 if the first weights and second weights of the medicine codes of the second code set are smaller than or equal to the cut-off value.

8. The apparatus of claim 7, wherein the second signal is received if the first code set is modified within a period of time.

9. The apparatus of claim 5, wherein, after updating the first weights and the second weights associated with the output medicine codes in the database in communication with the at least one processor, the apparatus further performs:
asserting the flag equal to 0.

10. The apparatus of claim 3, wherein updating the first weights and the second weights comprises on of the following one:
multiplying the first weights and the second weights by a number; and
weights comprises adding the first weights and the second weights with a number.

11. A method for processing a prescription, comprising:
receiving a first code set, the first code set comprising at least one diagnosis code and at least one medicine code;
outputting one or more medicine codes of the first code set;
receiving a first signal responsive to the output medicine codes; and
updating first weights and second weights associated with the output medicine codes in a database in communication.

12. The method of claim 11, wherein the first signal is received if the first code set is not modified within a period of time.

13. The method of claim 11, wherein the database includes a first weight between a diagnosis code and a medicine code and a second weight between a medicine code and another medicine code, and the first weight and the second weights are greater than zero.

14. The method of claim 13, wherein:
the output medicine codes has at least one first weight smaller than a cut-off value or has at least one second weight smaller than the cut-off value.

15. The method of claim 13, further comprising:
generating a flag;
setting the flag to 0;
comparing the total number of the first weights for the first code set greater than a cut-off value and the second weights for the first code set greater than the cut-off value to the number of medicine codes of the first code set;
setting a flag equal to 1 if the total number of the first weights for the first code set greater than a cut-off value and the second weights for the first code set greater than the cut-off value is smaller than the number of medicine codes of the first code set;
for each diagnosis code of the first code set, comparing the first weights to the cut-off value;
setting the flag equal to 1 if the first weights of the diagnosis codes of the first code set are smaller than or equal to the cut-off value;
for each medicine code of the first code set, comparing the first weights to the cut-off value and comparing second weights to the cut-off value; and
setting the flag equal to 1 if the first weights and second weights of the medicine codes of the first code set are smaller than or equal to the cut-off value.

16. The method of claim 15, further comprising:
if flag equals to 1, outputting one or more medicine codes of the first code set.

17. The method of claim 15, further comprising:
receiving a second signal responsive to the output medicine codes;
receiving a second code set comprising at least one diagnosis codes and at least one medicine codes;
setting the flag to 0;
comparing the total number of the first weights for the second code set greater than a cut-off value and the second weights for the second code set greater than the cut-off value to the number of medicine codes of the second code set;
setting a flag equal to 1 if the total number of the first weights for the second code set greater than a cut-off value and the second weights for the second code set greater than the cut-off value is smaller than the number of medicine codes of the second code set;
for each diagnosis code of the second code set, comparing the first weights to the cut-off value;
setting the flag equal to 1 if the first weights of the diagnosis codes of the second code set are smaller than or equal to the cut-off value;
for each medicine code of the second code set, comparing the first weights to the cut-off value and comparing second weights to the cut-off value; and
setting the flag equal to 1 if the first weights and second weights of the medicine codes of the second code set are smaller than or equal to the cut-off value.

18. The method of claim 17, wherein the second signal is received if the first code set is modified within a period of time.

19. The method of claim 15, further comprising:
after updating the first weights and the second weights associated with the output medicine codes in the database in communication with the at least one processor, asserting the flag equal to 0.

20. The method of claim 13, wherein updating the first weights and the second weights comprises one of the following:
multiplying the first weights and the second weights by a number; and
adding the first weights and the second weights with a number.
